(19) United States
(12) Patent Application Publication
(10) Pub. No.: US 2002/0031787 A1
(43) Pub. Date: Mar. 14, 2002

(54) Compositions and methods for reducing autoimmunity

(76) Inventors: Noel K. Maclaren, New York, NY (US); Anjli Kukreja, East Elmhurst, NY (US)

Correspondence Address: LAHIVE & COCKFIELD, 28 STATE STREET, BOSTON, MA, 02109, US

(21) Appl. No.: 09/775,687

(22) Filed: Feb. 2, 2001

Related U.S. Application Data
(60) Provisional application No. 60/180,305, filed on Feb. 4, 2000.

(51) Int. Cl. G01N 33/567

(52) U.S. Cl. 435/7.21

(57) ABSTRACT

Compositions and methods useful in the prevention or amelioration of autoimmune disorders, through immunostimulation of the innate component of the immune system are provided. In addition, the methods for the diagnosis or prediction of propensity to develop autoimmune disorders are provided, thus, permitting early treatment and, preferably, amelioration and prevention of such disorders reaching a clinical diagnosis. This stimulation of the innate arm of the immune response serves to improve or restore self-tolerance, thus, abrogating the autoimmune process.

### RELATED APPLICATIONS

This application claims priority to U.S. Ser. No. 60/180,305 filed on Feb. 4, 2000. The contents of that application are specifically incorporated herein by reference.

### BACKGROUND OF THE INVENTION

Autoimmune disorders cause a high degree of morbidity and mortality worldwide. Efforts to find a cure for autoimmune disorders have focused on elucidating the mechanism by which these diseases become established. An exemplary autoimmune disorder is immune mediated diabetes (IMD), a common subtype of insulin deficient or type-I diabetes. Much of the research into this disease has relied on the use of animal models, e.g., the non-obese diabetic (NOD) mouse. Autoimmune-mediated damage to insulin secreting pancreatic β cells begins in early in life in these mice as well as humans destined to develop the disease, although the pace of the disease and the time of clinical onset for persons so afflicted is quite variable. Autoimmunity proceeds as a chronic inflammatory process within the pancreatic islets, ultimately resulting in an absolute insulin deficiency and, thus, an inability to maintain normal levels of blood glucose. (Kukreja, A. and Maclaren, N. (1999). J Clin Endo Metab. 84: 4371- 4378; Atkinson, M. and Maclaren N. New Engl J Medicine (1992) 348-349; Atkinson, M. and Maclaren N. Scientific American 262(7) (1990): 61-71).

In diabetic subjects with IMD (as well as in NOD mice), there is evidence that T cells mediate autoimmune destruction of insulin secreting β cells. CD8+ cytotoxic T cells specific for islet cell protein antigens destroy the pancreatic cells directly by secretion of factors (e.g., perforin, certain cytokines, super-oxide radicals and/or enzymes, such as granzyme), or indirectly by the induction of apoptosis or programmed β cell death in the islet cells. This cellular pathological response in mice is indirectly affected by T helper (Th) cells of the Th1 phenotype, which secrete cytokines necessary for the development of cytotoxic T cells. Th1 cytokines include interleukin-2 (IL-2), tumor necrosis factors α and β (TNF -αand β) and interferon-gamma (IFN-γ). Th1 cytokines promote cellular immune responses, such as delayed hypersensitivity or cell-mediated cytolysis. Th2 cytokines, on the other hand, such as, IL-4, IL-5, IL-13 and IL-10 (Mossman, T. R. and Subash S. (1996). Immunol. Today 17: 138-146), promote humoral responses and, consequently, are important, in antibody-mediated responses (e.g., allergic reactions) for example (Kukreja, A. and Maclaren, N. (1999). J Clin Endo Metab. 84: 4371-4378; Atkinson, M. and Maclaren N. New Engl J Medicine (1992) 348-349; Atkinson, M. and Maclaren N. Scientific American 262(7) (1990): 61-71). In the case of IMD, while anti-islet Th1 responses are generally thought to be destructive, anti-islet Th2 responses are thought to be protective, acting to counter the Th1 responses that mediate IMD.

Polarization towards Th1 or Th2 dominated responses is controlled by the stimuli that instigate the response. In the development of an immune response, an antigen (classically a protein antigen) comes into contact with an antigen presenting cell (APC), such as a dendritic cell in tissues or a macrophage/monocyte in the blood. After a period of protein digestion, fragments (typically 9-12 amino acid sized peptides) are presented to Th cells in the context of class II major histocompatibility complex (MHC) antigens. These immunizing events are influenced by the cytokine milieu in which these events occur. For example, activated macrophages produce IL-12, which in turn stimulates the Th1 pathway. Other cytokine producing and regulatory cells, e.g., NK-T and CD25⁺ T cells also greatly influence these polarizing responses by secreting either Th1 or Th2 cytokines. For example, IL-12 of macrophage or NK-T cell origin drives immune responses towards the Th1 pathway. In contrast, IL-4 made by NK-T cells (or produced by Th2 cells in an ongoing Th2 response) drives developing responses towards the Th2 pathway.

There is an environmental component to autoimmune diseases such as IMD (Todd, J. A. Cell. (1996) 85:311-318). Although normal individuals have low numbers of autoreactive, circulating CD8+ T cells, such potentially autoreactive T cells can promote damaging autoimmune responses if they become activated. Such activation can occur, for example, by immunization with an antigen that mimics or cross-reacts with a self antigen expressed by islet cells. For example, infection with Coxsackie B virus can activate autoreactive T cells specific for glutamic acid decarboxylase expressed by pancreatic islet cells (Maclaren, N. and Atkinson M. (1997) Molecular Medicine 3 (2): 790-794]). This may be because there is a structural homology between the P2-C protein of the virus and an 18 mer sequence of the dominant isoforn of glutamic acid decarboxylase (GAD₆₅). In another example, a pancreotrophic virus (such as Coxsackie B) may induce an islet cell inflammation of a Th1 biased type through activation of macrophages and T cells invading the islets or by an immune response to islet cell antigens released by viral injury (Benoit, C. and Mathis, D. (1998). Nature 394:227-228; Howorwitz et al (1998). Nature Medicine 4: 781-785).

There is also a genetic component to autoimmune diseases such as IMD. In the case of IMD, a number of genes have been linked with the disease. The major genes are those encoding the Human Leucocyte Antigens (HLA) of the DR and DQ loci, on chromosome 6p. HLA-related susceptibility likely relates to the ability of the dendritic cells of the thymus which bear these HLA molecules to present self antigens with high affinity binding [Haung, W. and Maclaren, N. et al. J Clin Endo Metab (1996) 81 (7) 1-5; She, X., Immunology Today (1996) 17: 323-329. Another IMD gene is the regulator segment of the insulin (INS) gene. 11q. Upstream of this INS gene is an area of variable numbers of tandem repeats (VNTRs) and the protective allele is associated with increased expression of insulin itself on the dendritic cells. A third IMD gene encodes CTLA4, a molecule which is induced on activated T cells and which is capable of binding B7 antigens expressed by antigen presenting cells and down-modulating the T cell response by inducing apoptosis of activated T cells. The NOD mouse has an expanded lymphoid mass when compared with other mouse strains, suggesting that their T cells are defective in apoptosis. In human IMD subjects, genetic associations with CTLA-4 gene polymorphisms have also been reported (Morrow, M., Maclaren, N. and She, et al. Human Molecular Genetics (1998) (6) 8: 1275-1282).

Despite the fact that these environmental and genetic factors are known, diagnosing and treating autoimmune disorders has not been achieved using methods known in the prior art. Thus, there remains a clear need for compositions and methods for diagnosing and predicting the disease and for therapies that can reduce islet cell autoimmunity and prevent diabetes.

### SUMMARY OF THE INVENTION

The subject invention pertains to compositions and methods for the diagnosis of and use in the prevention or amelioration of autoimmune disorders. The instant invention is based on the finding that autoimmune diseases are in reality immuno-deficiency disorders; multiple defects in immune tolerance to self, permit immune attacks upon self (autoimmunities) to become established and perpetrate disease. Thus, where the immunosuppressive approaches of the prior art are likely to fail and be associated with significant side effects, immunostimulants that correct deficiencies underlying autoimmune diseases, although seemingly paradoxical, are useful in restoring tolerance to self. The claimed methods identify individuals at risk for autoimmune disease and reduce the symptoms of autoimmunity through immunostimulation of a subset of the innate component of the immune system, in particular immunoregulatory IL-2R or CD4+/CD25+ T cells and NK-T cells. This stimulation of the innate arm of the immune response serves to improve or restore self tolerance, thus, abrogating the autoimmune process.

Accordingly, the invention pertains to compositions and methods useful in activating cells that participate in immune regulation and thereby immune tolerance, i.e., natural killer like, thymus-derived lymphocytes (NK-T cells) and/or CD25⁺ T cells. Such cells are present in decreased numbers in many autoimmune disorders. In addition, their function also tends to be reduced in individuals with an ongoing autoimmune response. The activation of one or both of these cell types will have the effect of inducing control over the adaptive component of the immune system to minimize autoreactivity.

The subject compositions and methods improve or prevent autoimmune disorders by enhancing the activity and/or by increasing the numbers of these immunoregulatory T cells. This can be accomplished, e.g., by administering antigens to a subject, which result in stimulation of NK-T cells and/or CD25⁺ T cells.

These same cell populations have also been identified as being useful as indicator cells in the diagnosis and/or prediction of impending autoimmune disease like IMD, e.g., in mammalian subjects, preferably in human subjects.

Accordingly, in one aspect, the invention pertains to a method of predicting the propensity of a subject to develop an autoimmune disorder, by measuring i) the number or level of indicator T cells or ii) the activity of indicator T cells present in the subject as determinative of the propensity of a subject to develop an autoimmune disorder.

The invention further pertains to a method of diagnosing an autoimmune disorder by measuring i) the number or level of indicator T cells or ii) the activity of indicator T cells present in the subject in order to diagnose an autoimmune disorder.

The invention also pertains to a method of predicting the efficacy of treatment for an autoimmune disorder by measuring i) the number or level of indicator T cells or ii) the activity of indicator T cells present in the subject as determinative of the efficacy of treatment for an autoimmune disorder.

In one embodiment, the number or level of indicator T cells is measured using an antibody that recognizes T and NK-T cell surface markers selected from a group consisting of: i) an antibody that recognizes CD3 in combination with an antibody that recognizes at least one of CD69, CD94, or CD 161 and ii) an antibody that recognizes a TCR variable gene expressed region preferentially expressed by NK-T cells in combination with an antibody that recognizes at least one of CD69, CD94, or CD161.

In one embodiment, the antibody that recognizes a TCR variable region preferentially expressed by NK-T cells recognizes at least one of Vα24, Vβ11 or JαQ.

In one embodiment, the number or level of indicator cells is measured by detecting CD4+/CD25+ cells that do not express CD122 or CD132.

In another aspect, the invention pertains to a method of predicting the propensity of a subject to develop an autoimmune disorder by i) determining the number or level of indicator T cells in a biological test specimen, and ii) comparing the number or level of the indicator cells from the biological specimen to the number or level of the indicator cells in a control, wherein the presence of a reduced level of the indicator cells in the test specimen relative to the control is indicative of an increased propensity for the subject to develop an autoimmune disorder, to thereby predict the propensity of a subject to develop an autoimmune disorder.

In another embodiment, the invention pertains to a method of predicting the propensity of a subject to develop an autoimmune disorder by: i) contacting a biological specimen comprising indicator T cells obtained from a subject with one or more agents that stimulate cytokine production by the indicator cells, ii) determining the level of cytokines produced by the indicator cells, and iii) comparing the level of cytokines produced by the indicator cells to a control, wherein production of lower levels of cytokines by the indicator cells obtained from the subject is indicative of an increased propensity for the subject to develop an autoimmune disorder, to thereby predict the propensity of a subject to develop an autoimmune disorder.

In yet another aspect, the invention pertains to a method of determining the effectiveness of treatment for of autoimmune disorder by: i) determining the number or level of indicator T cells in the biological specimen obtained from a subject undergoing treatment for an autoimmune disorder, and ii) comparing the number or level of the indicator cells from the biological specimen to the number or level of indicator cells in a sample collected from the subject prior to treatment, wherein the presence of an increased number or level of indicator cells in the specimen from the subject is indicative of effectiveness of the treatment, to thereby determine the effectiveness of treatment for an autoimmune disorder.

In still another embodiment, the invention pertains to a method of determining the effectiveness of treatment for of autoimmune disorder by i) contacting indicator T cells in a post treatment biological specimen obtained from a subject undergoing treatment for an autoimmune disorder with one or more agents that stimulate indicator cell cytokine production, ii) determining the level of cytokines produced by the indicator cells, and iii) comparing the level of cytokines from the post treatment biological specimen from the subject to the level cytokines in a sample collected from the subject prior to treatment, wherein the presence of an increased level of cytotokines in the post treatment specimen is indicative of effectiveness of the treatment, to thereby determine the effectiveness of treatment for an autoimmune disorder.

In one embodiment, the cytokines are Th1 cytokines. In another embodiment, the cytokines are Th2 or TH3 cytokines.

In another aspect, the invention pertains to a method of preventing the development of an autoimmune disorder in a subject comprising, administration of an enhancing agent to the subject.

In one embodiment, the subject is known to be at risk for the development of an autoimmune disorder. In another embodiment, the subject is not known to be at risk for the development of an autoimmune disorder.

In still another aspect, the invention pertains to a method of ameliorating the symptoms of an ongoing autoimmune disorder in a subject comprising administering an enhancing agent to the subject.

In one embodiment, the enhancing agent is a bacterium or is a substance derived from a bacterium.

In one embodiment, the enhancing agent is administered orally.

In another embodiment, the enhancing agent is a bacterium from the genus Lactobacillus.

In another embodiment, the enhancing agent is derived from a bacterium belonging to a genus selected from the group consisting of: Mycobacteria, Bordatella, Corynebacterium, Streptococcus, or Hemophilus.

In another embodiment, the enhancing agent is administered orally.

In one embodiment, the enhancing agent is lipopolysaccharide.

In still another embodiment, the enhancing agent is in the form of a bacterial cell lysate.

In yet another embodiment, the enhancing agent is a purified or recombinant bacterial antigen.

In still another embodiment, the enhancing agent is lipo-arabinomannan (LAM).

In yet another embodiment, the enhancing agent is an α- galactosyl-ceramide.

In one embodiment, the autoimmune disorder is selected from the group consisting of: hay fever, allergic rhinitis, and asthma.

In yet another embodiment, the invention pertains to a kit for predicting the propensity of a subject to develop an autoimmune disorder or the effectiveness of a treatment for an autoimmune disorder comprising: at least one antibody which recognizes a cell surface marker on an indicator cell.

In one embodiment, the kit contains at least one antibody that recognizes a cytokine.

In one embodiment, the kit contains a means for isolating peripheral blood mononuclear cells.

### BRIEF DESCRIPTION OF THE DRAWINGS

FIG. 1 shows that CD3+ T cells in individuals either recently diagnosed with IMD, who had been developed IMD years before, or who are relatives of those individuals and are at risk for developing IMD are deficient in their expression of IFN-γ as well as IL-4

FIG. 2 shows that absolute numbers of NK-T (as measured by measuring the percentage of Vα24+ or Vβ11+ cells) cells are low in diabetic subjects.

FIG. 3 shows NK-T and T cell numbers and IFN-γ secretion in various subject groups. Newly diagnosed subjects were abnormal for both parameters.

FIG. 4 shows that when NOD mice are given either the diphtheria/pertussis/tetanus (DPT) or pneumococcal (pnu-immune) childhood vaccines, diabetes is clearly reduced as shown by actuarial or life table analyses.

FIG. 5 shows that the percentage of CD3⁺ cells that are Vα24⁺Vβ11⁺ NK-T cells is decreased in patients as compared to normal controls.

FIG. 6 shows that the percentage of CD3⁺IFNγ⁺ T cells is decreased in patients as compared to normal controls.

FIG. 7 shows the ratio of NK-T cells (as determined by reverse RT PCR analysis for mRNA for rearranged T cell receptors expressed by these cells compared to mRNA for a housekeeping gene) in mouse liver cells.

FIG. 8 shows the ratio of NK-T cells (as determined by reverse RT PCR analysis for mRNA for rearranged T cell receptors expressed by these cells compared to mRNA for a housekeeping gene) in mouse spleen cells.

FIG. 9 shows that the percentage of CD3⁺ cells that are CD4+/CD25⁺ T cells is decreased in patients as compared to normal controls.

FIG. 10 shows that the percentage of CD3⁺ cells that are CD4+/CD25⁺ T cells is decreased in patients as compared to normal controls.

### DETAILED DESCRIPTION OF THE INVENTION

The subject invention provides an advance over the prior art by providing compositions and methods useful in the prevention or amelioration of autoimmune disorders through immunostimulation using enhancing agents. These enhancing agents serve to improve or restore self-tolerance, preferably through stimulation of the innate arm of the immune response and, thereby abrogate the autoimmune process. In addition, the invention provides methods for the diagnosis or prediction of propensity to develop autoimmune disorders. Such early diagnosis and evaluation preferably permits early treatment and, preferably, amelioration of such disorders.

Before further description of the invention, the following terms are defined:

I. Definitions

As used herein, the term “autoimmune disease” or “autoimmune disorder” includes undesirable conditions that arise from an inappropriate or unwanted immune reaction against self-cells and/or tissues. The term “autoimmune disease or “autoimmune disorder” is meant to include such conditions, whether they be mediated by humoral or cellular immune responses. Exemplary autoimmune diseases or disorders include, but are not limited to: vitiligo, alopecia, rheumatoid arthritis, celiac disease, inflammatory bowel disease, chronic active hepatitis, Addison's disease, Hashimoto's disease, Graves disease, atrophic gastritis/pernicious anemia, acquired hypogonadism/infertility, hypoparathyroidism, and multiple sclerosis, Myasthenia gravis, Coombs positive hemolytic anemia, systemic lupus erthymatosis, chronic allergic diseases (such as asthma, hay fever, or allergic rhinitis), Siogren's syndrome, and immune mediated (type- 1) diabetes.

As used herein, the term “innate immunity” includes the arm of the immune response which, in contrast to the acquired arm of the immune response is not antigen specific does not show enhanced, secondary antigen-specific immune responses upon restimulation with the same antigen.

As used herein, the term “indicator cell” includes cells which serve as indicators of the autoimmune status of a subject, e.g., NK-T cells, CD4+/CD25⁺ T cells, and/or cytokine secreting T cells. Indicator cells are immunoregulatory T cells. As used herein, the term “activity” with respect to an indicator cell includes the ability of that cell to produce a cytokine.

As used herein, the term “NK-T cell” includes cytokine rich CD3⁺ T cells that usually do not express CD4 or CD8 in humans (i.e., are double negative). In some instances, NK-T cells are CD4 bearing cells (e.g., 50% of murine NK-T cells are CD4+). NK-T cells do express TCR αβchains. The αβchains these cells express are restricted in their variable gene chain repertoires; These cells express an invariant T cell receptor α chain (Vα24 in humans and Vα14 in mice) and a restricted, but polyclonal set of Vβ gene families (Vβ11 in humans and Vβ8, Vβ7 and Vβ2 in mice). NK-T cells respond to IL-12 by expressing high affinity IL-12 receptors, secreting IFN-γ, expressing markers such as NK1, CD69, CD94, and CD 161, and becoming competent to lyse tumor or NK cell targets. NK-T cells also produce IL-4 after cross-linking with anti-CD3 antibodies or TCR-mediated stimulation, particularly if they are immature. In one embodiment, NK-T cells express an additional marker, such as DX5 or an ortholog thereof (Moodycliffe, A. et. al. 2000 Nature Immunology. 1:521).

As used herein, the term “CD25⁺ T cells” includes T cells which are positive for CD4 and the α a chain of the IL-2 receptor, but which do not express either of the other chains of the IL-2 receptor, i.e., lack CD 122 (β chain) and CD132 (γ chain) expression. The CD25 subunit is present on a subset of resting thymocytes, but can also be induced in T cell activation.

As used herein, the term “enhancing agent” includes antigens, adjuvants, cytokines or other compounds which stimulate expansion of (or slow the reduction in levels of), promote the maturation of, and/or promote cytokine production by NK-T and/or CD4+/CD25⁺ T cells. In a preferred embodiment, enhancing agents comprise at least one lipid moiety, e.g., are lipid or lipo-polysaccharide antigens. Preferably, enhancing agents are recognized by NK-T cells in the context of CD1 molecules.

As used herein, the term “Th cytokine” includes “Th1,” “Th2,” and “Th3” cytokines. The term “Th1” cytokine includes cytokines that can be produced by ThI cells and that promote cell-mediated immune responses. Exemplary Th1 cytokines are known in the art and include: IL-2, IFN-γ, TNFα, and TNFβ. The term “Th2” cytokine includes cytokines that can be produced by Th2 cells and that promote humoral-mediated immune responses. Exemplary Th2 cytokines are also known in the art and include: IL-4, IL-5, IL-10, and IL-13. As used herein, the terms Th1 and Th2 cytokines refer to cytokines that can be produced by Th1 or Th2 cells and have been classified in this manner, regardless of the cell that produces them. For example, IL-4, although it can be produced by non-B, non-T cells, is a Th2 cytokine. The term “Th3” cytokine includes those cytokines that have been indicated in the art as being important in oral tolerance and downregulation of Th1 responses, e.g., TGFβ.

II. Diagnostic/Prognostic Methods

The instant invention provides methods for measuring the number or level of indicator cells in a subject or in a test sample obtained from a subject. If a subject that has not yet been diagnosed with an autoimmune disease is identified as having a reduced number or level of indicator cells or reduced indicator cell activity using the subject assays, that individual is identified as an individual likely to develop an autoimmune disease. If a subject previously diagnosed with an autoimmune disease is identified as having a reduced number or level of indicator cells or reduced indicator cell activity using the subject assays, that individual is identified as an individual likely to have a more severe disease course. If a subject diagnosed with an autoimmune disease who is undergoing treatment for that disease is identified as having a reduced number or level or activity of indicator cells or reduced indicator cell activity using the subject assays, that individual is identified as an individual that is a candidate for a modification of their current therapy regimen for the autoimmune disease. Any of the subjects described above having a reduced level of indicator cells can be treated using the treatment methods described herein.

These methods can be performed using a variety of art-recognized methods which assess the numbers or levels of indicator cells present in a subject or which assess the activity of such indicator cells.

The subject methods can be used in conjunction with methods of diagnosis or prognosis previously known in the art, e.g., detection of the presence of an antibody that is associated with an autoimmune disease or detection of a genetic marker associated with an autoimmune disease, or a family history of autoimmune disease.

In a preferred embodiment, the subject methods are used to identify a subject at risk for or developing immune mediated (type-1) diabetes. In another preferred embodiment, the subject methods are used to identify whether a subject has type-I diabetes or another form of the disease, such as type-2 diabetes.

Measurements of Indicator Cell Numbers or Levels

Absolute numbers or relative levels of cells (e.g., expressed as a percentage of a larger cell population) of cells that serve as indicators of the autoimmune status of the subject can be measured directly, e.g., by quantitating the absolute numbers or relative levels of such cells in vivo or in vitro. Numbers or levels of cells present in a subject can be detected (e.g., using a marker for such cells labeled with a detectable reagent (e.g., a radioactive tag whose presence and location in a subject can be detected by standard imaging techniques) or in vitro in a biological sample (e.g., a blood sample or biopsy) taken from a subject.

A preferred agent for detecting an indicator cell is an antibody capable of binding to surface marker expressed by that indicator cell, preferably an antibody with a detectable label such as a fluorescent dye. Antibodies can be polyclonal, or more preferably, monoclonal. An intact antibody, or a fragment thereof (e.g., Fab or F(ab′)₂) can be used. Another exemplary agent is a probe, e.g., that detects T cell receptor genes preferentially expressed by indicator cells. The term “labeled”, with regard to the probe or antibody, is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. Exemplary sites from which specimens can be collected include spleen, thymus, lymph node, liver, as well as the site of immune destruction in a particular autoimmune disorder.

In one embodiment, the presence of certain cell types in a biological sample can be detected based on their cell surface marker expression, e.g., using antibodies that stain for molecules expressed on these cells in combination with standard FACS or immunohistochemistry analysis. For example, in one embodiment, the numbers or levels of NK-T cells and/or CD4+/CD25⁺ cells in a sample is/are detected. Markers for detecting these cell types are known in the art. For example, CD25+ cells can be detected using fluorescence activated cell sorting (FACS) analysis to identify cells which stain positively for CD4 and CD25 and which stain negatively for CD122 and 132. NK-T cells can be detected, e.g., using antibodies which recognize the invariant TCR α chain or one of the restricted set of Vβ molecules expressed by such cells (in mice, NK-T cells have been found to preferentially use Vα14, while in humans Vα24JaQ chain receptors (usually in association with Vβ11) have been found to be preferentially used by NK-T cells). An antibody raised to the canonical TCR segment including VαQ, has given similar results by flow cytometry as that using both Vα24 and Vβ11 reactive antibodies. Alternatively, NK1 .1 cells can be detected by staining for cells expressing NK1.1 (or other NK activation markers, such as CD69, CD94, CD161 ) in conjunction with TCR or CD3. Such antibodies are commercially available and/or can be generated in the laboratory, e.g., using standard techniques, such as immunization of animals, phage display, and the like.

In another embodiment, NK-T cells can be detected using a molecular approach based on their restricted TCR α and β gene expression. For example, RNA can be isolated from cells, e.g., peripheral blood monocytes. in a biological specimen taken from a subject. cDNA, made from this RNA, can be amplified using two “nested” PCR cycles, e.g., Vα24 and Cα primers and a JαQ probe, to quantitatively detect expression of the TCR of interest using the TaqMan technology. The data can be expressed in absolute terms by simultaneous quantitation of a housekeeping (HPRT) gene. The use of PCR techniques to arrive at quantitative results has been described in the art and such quantitative assays can be used in the subject methods.

In one embodiment, numbers or levels of an indicator cell population from a subject (e.g., present in a test biological specimen taken from the subject) are compared to the levels of the same indicator cell population in a control sample.

Measurements of Indicator Cell Activity

In addition to, or instead of, measuring numbers or levels of indicator cells, the activity of indicator cells can be measured, e.g., by determining levels of cytokines secreted by such cells. For example, NK-T cells can produce Th1-type and Th2-type cytokines. Accordingly, either or both of these classes of cytokines can be measured using methods that are well known in the art. In one embodiment, cytokines can be measured in vivo. In another embodiment, cytokines can be measured in vitro, e.g., by removing a biological sample comprising indicator cells from a subject and stimulating the cells in vitro and measuring the amount or levels of cytokines produced. Such cytokine levels can be used to determine the activity of an indicator cell population in a subject. Techniques for measuring cytokine production are known in the art and include, e.g., ELISPOT assays, flow cytometry assays, ELISA assays, and PCR.

In addition, a global T cell defect in cytokine secretion has been correlated with the propensity to develop autoimmune disease. Thus, in one embodiment, T cells can serve as indicator cells in the subject methods. Accordingly, in one embodiment, a biological sample comprising T cells can be tested for their activity to identify a subject with a reduced number or level or activity of indicator cells. When the activity of T cells is to be measured, polyclonal activators of T cell cytokine production can be used, e.g., the combination of phorbol myristate acetate (PMA) + ionomycin, an anti-CD3 antibody, or a superantigen.

Kits for Determination of Indicator Cell Numbers or Levels and/or Indicator Cell Activity

The invention also encompasses kits for detecting the presence of and/or activity of indicator cells in a biological sample. For example, the kit can comprise one or more of: a labeled compound or agent capable of detecting a cytokine produced by an indicator cell or a molecule expressed on the surface of an indicator cell or a nucleic acid molecule present in an indicator cell, and a means for comparing sample data with a control or standard. The compound or agent, e.g., antibodies or probes as described above, can be packaged in a suitable container. The kit can further comprise other agents that would aid in the diagnosis or prognosis of an autoimmune disorder. The kit can further comprise instructions for using the kit to determine the number or level of indicator cells in a sample.

Subject populations

As set forth briefly above, the number or level or activity of indicator cells can be tested in a variety of subject populations using the methods provided herein. For example, in one embodiment of the invention, the nunber or level or activity of indicator cells can be tested in an individual that is at risk for the development of an autoimmune disorder. For example, such a subject may have some genetic predisposition to develop an autoimmune disorder, e.g., based on family history or previous positive diagnostic result.

In another embodiment, a subject that may be positive for some other indicator of the disease may be tested using the claimed methods. For example, in the case of IMD, a subject who has not yet developed the disorder may be positive for islet cell autoantibody markers and may be at risk for or in process of developing IMD (Maclaren et al. (1999) J Autoimmunity 12 94) :279-287; Riley, W., Maclaren, N. et al (1990) New England J Medicine 323: 1167-1172). Exemplary autoantibody markers can be, e.g., (i) components of the islet cell autoantibody (ICA) reaction seen by fluorescence microscopy, such as antibodies described by Anstoot H K, et al. ((1997) J Clin Invest 97: 2772-2783); antibodies to the lower molecular weight isoform of two glutamic acid decarboxylases (GAD) (Kauffman, D., Maclaren, N. et al. J Clin. Invest 89:283-292); or two forms of the transmembrane tyrosine phosphatases (Lu, J., et al. Proc Natl Acad. Science USA, (1996) 93: 2307-2311; Lan, M., et al. (1996) Proc. Natl Acad Science USA 93: 6367-6370)) or (ii) autoantibodies to insulin (Atkinson, M. Maclaren, N. et al (1986) Diabetes 35: 894-898). Relatives of subjects with IMD, are at high risk to develop IMD themselves if there has been epitope spreading manifested by presence of more than one of these autoantibodies.

Subjects with an inherited predisposition to develop IMD because of their high risk HLA-DR/DQ genotypes in the major histocompatibility complex on chromosome 6p, VNTR alleles 5′ to the insulin gene on chromosome 11q and high risk CTLA-4 genes on chromosome 2q are also candidates for this approach. Since IMD is considered to be a Th1 mediated auto-immune disease, individuals with the auto-immune poly-glandular syndromes (APS) which include IMD (APS-2 and APS-3) can also be tested using the claimed methods.

In another embodiment, the number or level or activity of indicator cells can be tested in an individual that is not known to be at risk for the development of an autoimmune disorder, e.g., in a random screening test.

In another embodiment, the number or level or activity of indicator cells can be tested in an individual that has been newly diagnosed with an autoimmune disorder to correctly identify the type of the disease, and thereby predict the severity of the disorder and/or to assist in developing a treatment protocol. For example, subjects with positive islet cell autoantibodies who have newly diagnosed diabetes can be diagnosed as having the immune mediated form of type 1 diabetes (IMD), which can be similarly tested using the claimed methods (Neufeld M, Maclaren N et al. (1980) Diabetes 29: 589-594).

III. Enhancing agents

In another aspect, the invention pertains to methods of stimulating the innate arm of the immune response by administration of enhancing agents. Individuals (e.g., individuals as described above in the diagnostic/prognostic methods section) or cells from such individuals, can be treated with enhancing agents.

Enhancing agents for use in the claimed methods stimulate the innate limb of the immune system. Preferably, such enhancing agents stimulate cytokine production by indicator cells. Preferably, the administration of such enhancing agents results in stimulation of NK-T and or CD25+ T cell responses in autoimmune subjects, e.g., by improving their function and/or by increasing their numbers. Administration of enhancing agents to a subject with an ongoing autoimmune response preferably results in cytokine production profiles that more closely resemble those of a normal (non-autoimmune) subject. In a preferred embodiment, enhancing agents stimulate cytokine secretion by NK-T cells.

In one embodiment, enhancing agents comprise lipid or glyco-lipid moieties.

In another embodiment, enhancing agents are presented in the context of CD-1 molecules.

The subject enhancing agents may be synthetic or naturally occurring.

In one embodiment, an enhancing agent for use in the claimed methods is a naturally occurring molecule or is derived therefrom. Preferably, an enhancing agent comprises or is derived from a microbe(s), such as a bacterium and/or parasite (including multicellular parasites such as helminths or nematodes)) or a population thereof. In one embodiment, the enhancing agent is a viable microbe(s) (e.g., an attenuated form) which is administered to a subject. Such microbes can be administered via a number of routes (described in more detail below) or can be administered as a living vaccine to colonize the subject resulting in colonization of the subject and a change in the intestinal flora. Such microbes can also be administered parenterally.

Exemplary microbes include gram positive or gram negative bacteria. Exemplary bacteria include those from the genera: Mycobacterium, Proprionibacterium, Lactobaccilus, Bordatella, Corynebacterium, Streptococcus, and Hemophilus. Preferred lactobacilli include: L. plantarum, L. rhamnosus and L. paracasei.

Enhancing agents can also be derived from microbial cells. For example, molecules on the cell surfaces of gram-negative or gram positive bacteria can be used. Exemplary molecules include peptidoglycans, lipoteichoic acid and endogenous lipopolysaccharides as well as other components. For example, Lipoarabinomannan (LAM) derived from M. bovis can be used.

Other exemplary enhancing agents include bacterial antigens such as endotoxin or lipopolysaccharide. In one embodiment, an enhancing agent for use in the claimed methods can be a synthetic antigen.

For example, compounds comprising an α-galactosylceramide or the like can be synthesized for use in the claimed methods using techniques known in the art (e.g., using a general chemical synthesis method as for sphingoglycolipid (e.g, as described in Agricultural and Biological Chemistry. 1990.54:663) or as described in Liebigs Annalen der Chemie. 1988. p. 663. or in U.S. Pat. 6,017,892 or 6,071,884) Alternatively, α-galactosylceramide can be derived from sphingosine using various chemical modifications known in the art.

Other exemplary enhancing agents include 2-phenyl-1,2-benzoisoseranazol-3(2H); muramyldipeptide derivatives (WO/01778).

In one embodiment, an enhancing agent is a compound of the formula (I):

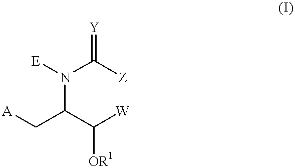

wherein:

A is a sugar moiety;

E is hydrogen, substituted or unsubstituted alkyl, unsubstituted or substituted alkenyl, substituted or unsubstituted alkynyl, or substituted or unsubstituted acyl;

Y is oxygen or sufur;

Z and W are each independently selected chain moieties;

R¹ is hydrogen or an hydroxyl prodrug moiety; and pharmaceutically acceptable salts thereof.

In one embodiment, the sugar moiety is a monosaccharide (e.g., a pentose or a hexose).

In one embodiment, the hexose is selected from the group consisting of allose, altrose, glucose, mannose, gulose, idose, galactose, talose, and derivatives thereof.

In one embodiment, E is hydrogen. In one embodiment, E is a lower alkyl. In one embodiment, Y is oxygen.

In one embodiment, Z is a straight or branched chain of one to thirty atoms. In one embodiment, the chain is substituted or unsubstituted alkyl. In another embodiment, the chain is substituted with one or more hydroxyl groups. In one embodiment, the chain is substituted or unsubstituted alkenyl.

In another embodiment, W is a straight or branched chain of one to thirty atoms. In another embodiment, a chain is substituted or unsubstituted alkyl. In one embodiment, the chain is substituted with one or more hydroxyl groups. In another embodiment, the chain is substituted or unsubstituted alkenyl.

In one embodiment, R¹ is hydrogen.

In another embodiment, R¹ is a hydroxyl prodrug moiety.

In another embodiment, the enhancing agent is a compound of formula II:

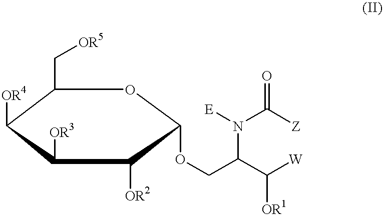

wherein:

R¹ , R², R³, R⁴, and R⁵ are each independently hydrogen or a hydroxyl prodrug moiety;

E is hydrogen, substituted or unsubstituted alkyl, unsubstituted or substituted alkenyl, substituted or unsubstituted alkynyl, or substituted or unsubstituted acyl;

Z and W are each independently selected chain moieties; and pharmaceutically acceptable salts thereof.

In one embodiment, R¹ is hydrogen.

In another embodiment, one or more of R², R³, R⁴, and R⁵ are hydrogen.

In still another embodiment, each of R², R³, R⁴ and R⁵ are hydrogen.

In another embodiment, E is hydrogen.

In yet another embodiment, Z is a substituted or unsubstituted chain of one to thirty atoms.

In another embodiment, Z is a chain of carbon atoms.

In one embodiment, chain is substituted with one or more substituents. In one embodiment, at least one of said substituent is hydroxyl or lower alkyl.

In one embodiment, said chain is alkyl.

In another embodiment, the chain is alkenyl.

In yet another embodiment, the chain is of the formula:

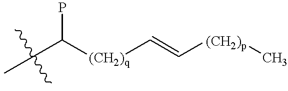

wherein

P is hydrogen or hydroxyl;

q is an integer from 0 to 27; and

p is an integer from 0 to (27-q).

In one embodiment, P is hydrogen.

In another embodiment, P is hydroxyl.

In another embodiment, q is 3, 4, 5, 6, 7, 8, 9, 10, 11, or 12.

In one embodiment, q is 6, 7, or 8.

In one embodiment, q is 7.

In one embodiment, p is 3, 4, 5, 6, 7, 8, 9, 10, 11, 12, 13, or 14.

In one embodiment, p is 6, 7, or 8.

In one embodiment, p is 7.

In one embodiment, W is a chain of one to twenty atoms.

In one embodiment, the chain is a chain of carbon atoms.

In one embodiment, the chain is substituted or unsubstituted alkyl.

In one embodiment, chain is substituted with one or more hydroxyl groups.

In one embodiment, the chain is (CH₂)₄₋₁₈CH₃; (CHOH)(CH₂)₅₋₁₈CH₃; or (CHOH)(CH₂)₅₋₁₈CH(CH₃)₂.

In another embodiment, the enhancing agent is a compound of the formula III:

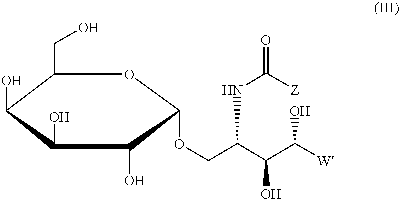

wherein

Z is a chain of 15 to 30 carbon atoms;

W' is chain of 8 to 15 carbon atom; and pharmaceutically acceptable salts thereof.

In one embodiment, Z is substituted or unsubstituted alkyl.

In another embodiment, Z is unsubstituted alkyl.

In still another embodiment, W' is substituted or unsubstituted alkyl.

In yet another embodiment, W' is unsubstituted alkyl.

Other enhancing agents can be identified based on assaying their ability to stimulate the innate immune system. For example, peripheral blood NK-T cells from subjects (e.g., control subjects) can be obtained. NK-T cell clones can be generated, e.g., by MACSorting with use of antibodies to NK-T cell markers. Cells can be cloned and maintained in culture (e.g., in medium such as RPMI 1640 with supplements and a growth factor such as recombinant IL-2). The cells can be expanded e.g., with IL-2 and restimulated every 2 weeks using a polyclonal T cell activating agent (e.g., phytohemagglutinin (PHA)) and a growth factor, e.g., IL-2, in the presence of irradiated feeder cells (e.g., irradiated at 1,500 to 3,000 rads). The purity of the resulting cells can be verified, e.g., using fluorescence activated cell sorting (FACS) (for example, staining cells with antibodies that recognize the V regions preferentially used by NK-T cells).

Clones obtained using this, or another art recognized method, can be incubated with candidate enhancing agents in culture in the presence of transgenic antigen presenting cells expressing CD1a or CD1d molecules (e.g., mammalian cells such as CHO cells trasfected with human transfected CD1a or CD1d molecules). Proliferative responses of the NK-T cells clones can be measured by ³H thymidine uptake and/or cytokine responses of NK-T cells can be measured, e.g., the production of IFN-γ and/or IL-4 can be detected by FACS analysis or by assaying for the release of cytokines into the tissue culture media using art-recognized techniques (e.g., bioassays or ELISA assays).

A composition comprising an enhancing agent of the invention may contain other additional agents. Such additional agents may be included in the composition, e.g., to produce a synergistic effect with the enhancing agent, or may be included to amelioriate symptoms of the autoimmune disease.

For example, in one embodiment, a vaccine or an immunogen may be administered in combination with an enhancing agent. In one embodiment, a composition can include an adjuvant, such as alum, Freund's complete or incomplete adjuvant, or similar immunostimulatory agent. In another embodiment, an antigen associated with an autoimmune disease can be administered. For example, in the case of IMD, islet cell antigens (such as insulin and glutamic acid decarboxylase (GAD₆₅), either alone or in combination) can be administered in combination with an enhancing agent.

Enhancing agents (and/or additional agents) can be administered via any appropriate route to a subject. In one embodiment, the enhancing agent and/or any additional agents can be administered via routes which promote Th2 responses (such as intra-nasally or by parenteral immunizations in adjuvants). In another embodiment, such antigens can be administered with a cytokine that promotes the development of Th2 responses, e.g., IL-4 or Th3 responses eg TGF-β.

IV. Pharmaceutical Compositions

Enhancing agents (active compounds) of the invention can be incorporated into pharmaceutical compositions suitable for administration. Such compositions typically comprise the enhancing agent and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the subject compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The pharmaceutical composition of the invention may be in the form of a liposome in which isolated enhancing agent is combined, in addition to other pharmaceutically acceptable carriers, with amphipathic agents such as lipids which exist in aggregated form as micelles, insoluble monolayers, liquid crystals, or lamellar layers which in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N..J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In certain embodiments, prevention of the action of microorganisms can be achieved by various antibacterial and antifingal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the enhancing agent in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdernal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For a compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

In practicing the method of treatment or use of the present invention, a therapeutically effective amount of an enhancing agent is administered to a subject. An enhancing agent may be administered in accordance with the method of the invention either alone or in combination with other therapies or agents. When co-administered with one or more other e agents, the enhancing agent and such additional agent(s) may be administered together or separately, either simultaneously with the other agent(s), or sequentially. If administered sequentially, the attending physician will decide on the appropriate sequence of administering the enhancing agent in combination with other factors or agent(s).

As used herein, the term “therapeutically effective amount” means the total amount of each active component of the pharmaceutical composition or method that is sufficient to show a meaningful subject benefit, e.g., production of desired effect, amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously. Preferably, a therapeutically effective amount of an enhancing agent is administered to a subject.

Administration of enhancing agents used in the pharmaceutical composition or to practice the method of the present invention can be carried out in a variety of conventional ways, such as oral ingestion, inhalation, or cutaneous, subcutaneous, or intravenous injection.

When a therapeutically effective amount of an enhancing agent is administered orally, the agent can be in the form, e.g., of a tablet, capsule, powder, solution, elixir, or the like. When administered in tablet form, the pharmaceutical composition of the invention may additionally contain a solid carrier such as a gelatin or an adjuvant. The tablet, capsule, and powder contain from about 5 to 95% enhancing agent, and preferably from about 25 to 90% enhancing agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as 5 ethylene glycol, propylene glycol or polyethylene glycol. When administered in liquid form, the pharmaceutical composition contains from about 0.5 to 90% by weight of enhancing agent, and preferably from about 1 to 50% enhancing agent.

When a therapeutically effective amount of enhancing agent is administered by intravenous, cutaneous or subcutaneous injection, the enhancing agent is preferably in the form ofa parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection can contain, in addition to the enhancing agent an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additive known to those of skill in the art.

The practice of the present invention will employ, unless otherwise indicated, conventional techniques of cell biology, cell culture, molecular biology, genetics, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, N.Y. (1995)); DNA Cloning, Volumes I and II (D. N. Glover ed., 1985); Oligonucleotide Synthesis (M. J. Gait ed. (1984)); Mullis et al U.S. Pat. No: 4,683,195; Nucleic Acid Hybridization (B. D. Hames & S. J. Higgins eds. (1984)); the treatise, Methods In Enzymology (Academic Press, Inc., N.Y); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The contents of all references, pending patent applications and published patents, cited throughout this application are hereby expressly incorporated by reference.

The invention is further illustrated by the following examples, which should not be construed as further limiting.

### EXAMPLES

### Example 1

Peripheral blood T cells were obtained from subjects who had either recently developed clinical IMD, had developed IMD years before, or who were relatives who were positive for islet cell autoantibodies (ICA) with (high risk) and without (lower risk) additional autoantibodies such as to insulin, GAD₆₅ or IA-2.

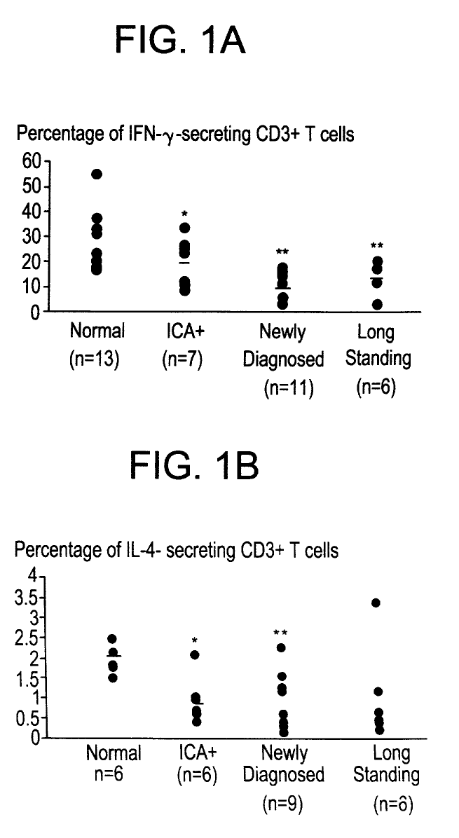

The peripheral blood mononuclear cells (PBMCs) of subjects and controls were stimulated with the polyclonal activators phorbol myrystate acetate (PMA) plus calcium ionomycin (I), which stimulate T cell proliferation through activation of protein kinase C (PKC) and calcium flux. The results are shown in FIG. 1. Compared to the normal controls, CD3+ T cells in subjects were deficient in their expressions of IFN-γ (a Th1 cytokine) as well as IL-4 (a Th2 cytokine) by flow cytometry analyses. However, the deficiency was more evident in the secretion of IFN-γ. The results suggest a global T cell defect involving cytokine secretion.

### Example 2

Low Numbers of Circulating NK-T Cells were found in Subjects with IMD at all Stages of their Disease.

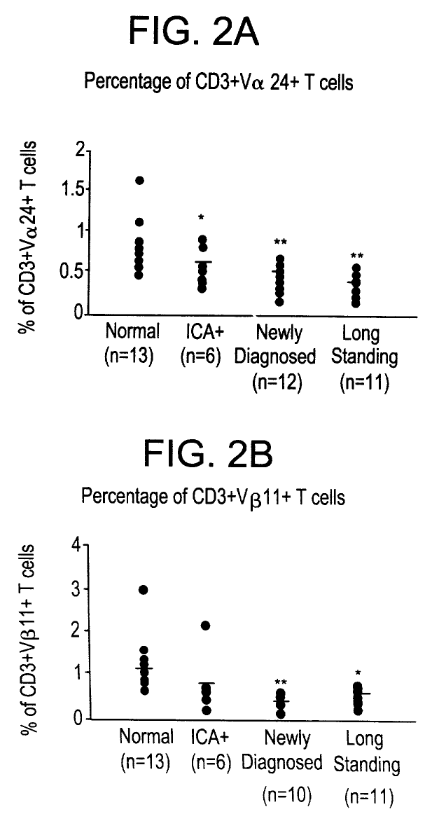

The results of this example are shown in FIG. 2. The absolute numbers of NK-T cells in samples from subjects as determined by flow cytometry by antibodies to their restricted TCR expression were conspicuously low. Both newly diagnosed and long standing diabetic subjects had similarly decreased numbers of NK-T cells as compared to normal control levels. The numbers of NK-T cells from relatives of subjects with IMD and relatives who were autoantibody positive but without diabetes when studied, were generally low as determined by measuring the percentage of CD3⁺/Vα24 ⁺ T cells or the percentage of CD3⁺Vβ11⁺ T cells. However, one relative with ICA only (without the other antibodies) was normal.

Families of the subjects with IMD have been studied and multiple members of some of these families have been found to suffer from other autoimmune disorders. These disorders included inflammatory bowel disease, multiple sclerosis, systemic lupus erythematosis. All had low NK-T cells also. This strongly suggests that all of these autoimmune disorders have a common NK-T cell lesion.

### Example 3

Low numbers of NK-T cells Correlates with Defective Ability of T cells to Secrete Cytokines Normally After PMA + Ionomycin.

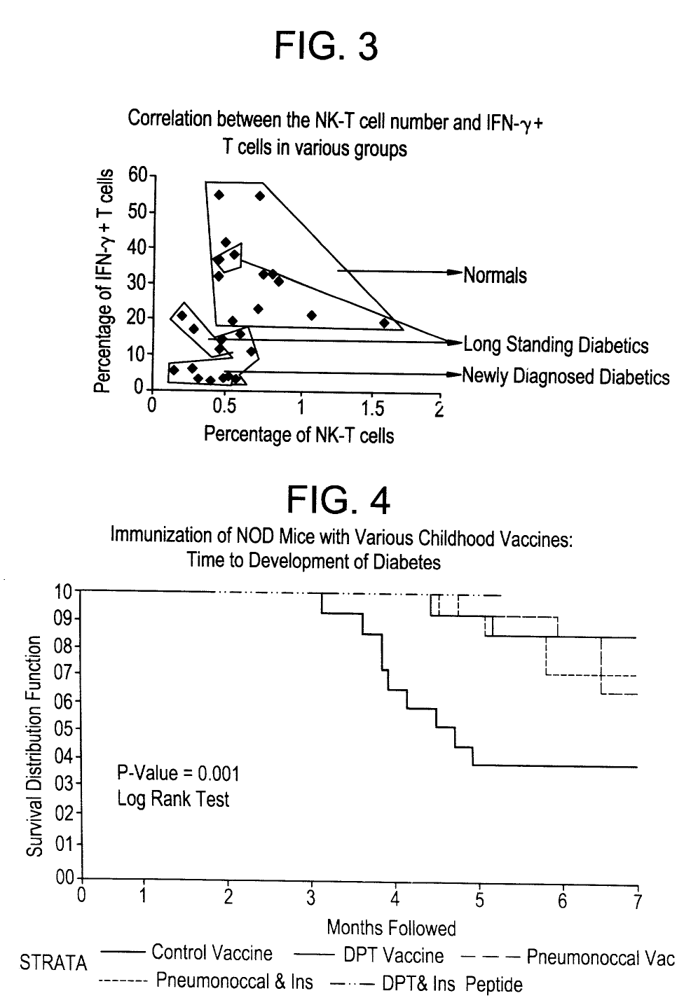

Both T cell and NK-T cell numbers were assessed in subjects. Newly diagnosed subjects are clearly abnormal for both parameters, as were most of the subjects with long standing IMD. Interestingly, in normal controls there was a curvilinear relationship between these parameters. Subjects were clearly separable from controls when both were considered with little overlap. However, two long standing subjects had low levels of NK-T cells but showed normal cytokine response to PMA + Ionomycin, as shown in FIG. 3. Seven ICA+ relatives were studied, of which two had additional autoantibodies to GAD₆₅ or IA-2 and five did not. Both of the subjects with multiple autoantibodies (who are at high risk of progressing to overt diabetes) had low levels of NK-T cells and defective T cell cytokine responses to PMA + Ionomycin stimulation, while the remainder had comparably lower responses than controls, indicating the utility of these measures in diagnosis and in disease prediction. This reduction in cytokine secretion has been observed for both IFN-γ(a Th1-type cytokine) and IL-4 (a Th2 type cytokine).

### Example 4

Prevention of Diabetes in NOD Mice given Bacterial Adjuvants:

When NOD mice are given either the diphtheria/pertussis/tetanus (DPT) or pneumococcal (pnu-immune) childhood vaccines, diabetes was clearly reduced as shown by actuarial or life table analyses in FIG. 4. Thus, the bacterial antigen based therapies protecting against diabetes likely do so by acting on NK-T cells to stimulate them, since such cells respond to bacterial antigens of a lipid nature and we have determined that the levels of NK-T cells in NOD mice is very low (see below). The effect was enhanced when insulin B chain 8-20 amino acid peptide was added. This islet cell antigen is important in the pathogenic sequence as we have shown before when the peptide was given in context of an adjuvant like incomplete Freund's adjuvant (Ramiya V. K, Maclaren N et al. (1997) J Autoimmunity 10: 287-292; U.S. Pat. 5,891,435).

### Example 5

Low Numbers of Circulating NK-T Cells were found in over 80 Subjects with IMD.

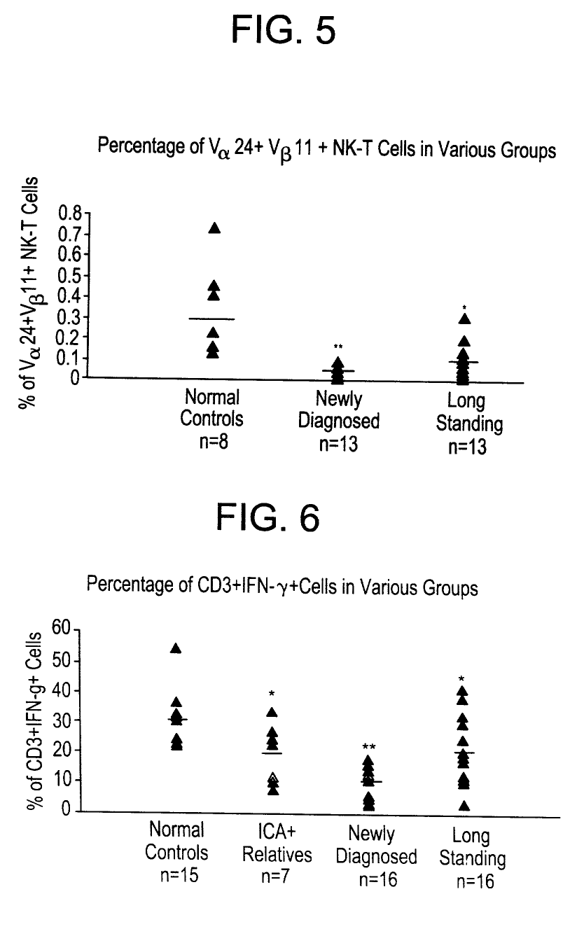

More than eighty subjects with immune mediated (type 1) diabetes and normal matched controls have been studied, and all with unequivocal new onset type-1 diabetes with positive islet cell autoantibodies have been found to have low numbers of NK-T cells as measured by flow cytometry, using conjugated antibodies against their canonical T cell receptors comprising Vα24-Vβ11 segments. The results of such an experiment are shown in FIG. 5 which shows the percentage of CD3⁺ cells that are Vα24⁺Vβ11⁺ NK-T cells in various patient groups as measured by FACs analysis. Proof that these cells are NK-T cells and not normal T cells that happen to contain the segment has been proven using an antibody that is directed against the Vα24-JαQ interface, with essentially identical results. Further, using a molecular (quantitative RT-PCR) approach to quantify the same canonical TCR transcripts with a PCR based method using a Vα24 primer and a JαQ probe (TaqMan), the results first obtained from flow cytometry were confirmed. mRNA analysis revealed that CD4+ cells from PBMCs obtained from a normal person do not express significant levels of Vα24-JαQ. However, double negative cells or unfractionated PBMCs from the same normal person show high expression of Vα24-JαQ. Unfractionated PBMCs from type-1 diabetes patients do not show significant expression of Vα24-JαQ. By these types of analysis, it has been confirmed that type -1 diabetes patients have only about 10% of the normal number of NK-T cells. In addition, as shown in FIG. 6, the percentage of CD3⁺IFNγ+ T cells in various patient groups has also been shown to be lower than the percentage of these cells in normal controls. These data further demonstrate that NK-T cells are low in numbers in immune mediated type-1 diabetes either before, at the time of and after clinical onset of diabetes.

### Example 6

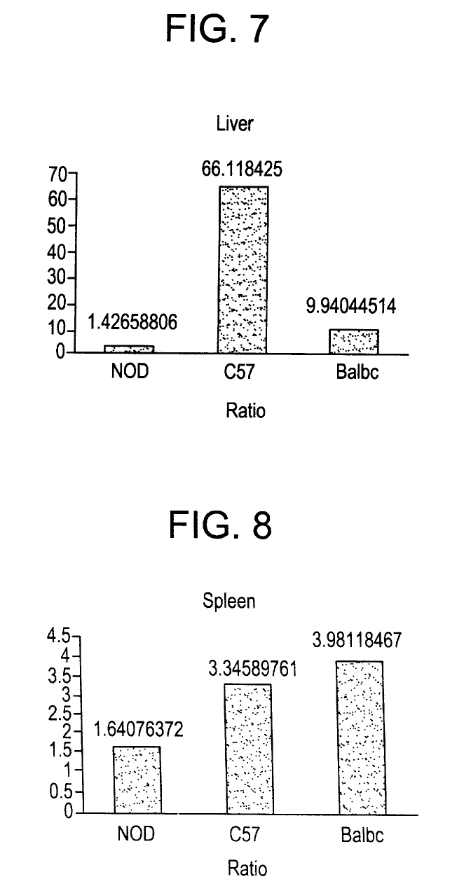

Low Numbers of NK-T Cells were found in NOD mice. Non-obese diabetic (NOD) mice were studied and found to have a striking deficiency in peripheral cells (<10% of the numbers of C57BLK/s hepatic T cells and <15% of normal BALB/c mice). FIG. 7 compares the NK-T cell ratio (as measured by reverse RT PCR to measure of rearranged TCR genes as compared to expression of a housekeeping gene) in liver cells from these mice and FIG. 8 compares the NK-T cell ratio in spleen cells from these mice. These experiments were done using the same (quantitative RT-PCR) molecular approach for determination of mRNA for the murine canonical TCR segment containing the Vα14-Jα281 as outlined above. Splenocytes were also reduced in NOD mice compared to these same mice while thymocyte levels of canonical TCR expression were similar between all 3 strains. These findings document that the major spontaneous diabetic model of human type-1 diabetes shares the same regulatory NK-T cell deficit.

### Example 7

Low numbers of CD25+ CD4+ T cells in subjects with autoimmunity

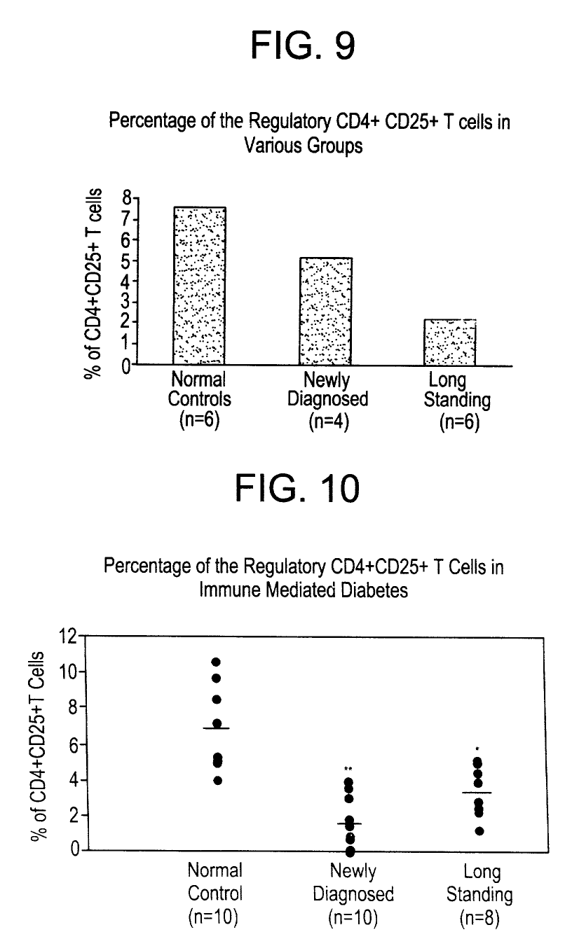

T cells that express interleukin-2 (IL-2) receptors have been implicated as regulatory cells important to immune tolerance. In a mouse model of autoimmunity, normal mice develop autoimmune diseases when they undergo thymectomies at 3 days of age. Lymphocytic infiltrates of the gastric mucosa, ovaries, thyroid, and sometimes the myocardium occur. When numbers of CD4+/CD25⁺ T cells are detected by antibodies that detect expression of IL-2Rαchains are quantitated in these mice, they are found to be reduced in number as compared to control mice. When such cells are given back to such mice, autoimmunity is abrogated (Suri-Payer et al. 1998. J Autoimmunity. 160:1212). FIGS. 9 and 10 show that these same regulatory T cells are present in lower numbers in subjects (expressed here as a percentage of CD3⁺ cells) with human immune mediated (type 1) diabetes.

What is claimed is:

1. A method of predicting the propensity of a subject to develop an autoimmune disorder, comprising measuring i) the number or level of indicator T cells or ii) the activity of the indicator cells present in the subject as determinative of the propensity of a subject to develop an autoimmune disorder.

2. A method of diagnosing an autoimmune disorder comprising, measuring i) the number or level of indicator T cells or ii) the activity of the indicator cells present in the subject in order to diagnose an autoimmune disorder.

3. A method of predicting the efficacy of treatment for an autoimmune disorder comprising, measuring i) the number or level of indicator T cells or ii) the activity of the indicator cells present in the subject as determinative of the efficacy of treatment for an autoimmune disorder.

4. The method of any of claims 1-3, wherein the number or level of indicator T cells is measured using an antibody that recognizes T and NK-T cell surface markers selected from a group consisting of: i) an antibody that recognizes CD3 in combination with an antibody that recognizes at least one of CD69, CD94, and CD 161; ii) an antibody that recognizes a TCR variable gene expressed region preferentially expressed by NK-T cells in combination with an antibody that recognizes at least one of CD69, CD94, and CD 161; and iii) an antibody that recognizes a TCR variable gene expressed region preferentially expressed by NK-T cells in combination with an antibody that recognizes CD3 and an antibody that recognizes at least one of CD69, CD94, and CD161.

5. The method of claim 4, wherein the antibody that recognizes a TCR variable region preferentially expressed by NK-T cells recognizes Vα24 and Vβ11 and JαQ.

6. The method of any of claims 1-3, wherein the number or level of indicator cells is measured by detecting CD4+/CD25+ T cells that are CD122 or CD132 negative.

7. A method of predicting the propensity of a subject to develop an autoimmune disorder comprising: i) determining the number or level of indicator T cells in a biological test specimen, obtained from the subject, and ii) comparing the number or level of the indicator cells from the biological specimen to the number or level of the indicator cells in a control, wherein the presence of a reduced level of the indicator cells in the test specimen relative to the control is indicative of an increased propensity for the subject to develop an autoimmune disorder, to thereby predict the propensity of a subject to develop an autoimmune disorder.

8. A method of predicting the propensity of a subject to develop an autoimmune disorder comprising:
i) contacting a biological specimen comprising indicator T cells obtained from a subject with one or more agents that stimulate cytokine production by the indicator cells,
ii) determining the level of cytokines produced by the indicator cells, and
iii) comparing the level of cytokines produced by the indicator cells to a control, wherein production of lower levels of cytokines by the indicator cells obtained from the subject is indicative of an increased propensity for the subject to develop an autoimmune disorder, to thereby predict the propensity of a subject to develop an autoimmune disorder.

9. A method of determining the effectiveness of treatment for of autoimmune disorder comprising:
ii) determining the number or level of indicator T cells in the biological specimen obtained from a subject undergoing treatment for an autoimmune disorder, and
ii) comparing the number or level of the indicator cells from the biological specimen to the number or level of indicator cells in a sample collected from the subject prior to treatment, wherein the presence of an increased number or level of the indicator cells in the specimen from the subject is indicative of effectiveness of the treatment, to thereby determine the effectiveness of treatment for an autoimmune disorder.

10. A method of determining the effectiveness of treatment for of autoimmune disorder comprising:
i) contacting indicator T cells in a post treatment biological specimen obtained from a subject undergoing treatment for an autoimmune disorder with one or more agents that stimulate indicator cell cytokine production,
ii) determining the level of cytokines produced by the indicator cells, and
iii) comparing the level of cytokines from the post treatment biological specimen from the subject to the level cytokines in a sample collected from the subject prior to treatment, wherein the presence of an increased level of cytotokines in the post treatment specimen is indicative of effectiveness of the treatment, to thereby determine the effectiveness of treatment for an autoimmune disorder.

11. The method of any of claims 1-3, wherein the cytokines are Th‘cytokines.

12. The method of any of claims 1-3, wherein the cytokines are Th2 or TH3 cytokines.

13. A method of preventing the development of an autoimmune disorder in a subject comprising, administering an enhancing agent to the subject.

14. The method of claim 13, wherein the subject is known to be at risk for the development of an autoimmune disorder.

15. The method of claim 13, wherein the subject is not known to be at risk for the development of an autoimmune disorder.

16. A method of ameliorating the symptoms of an ongoing autoimmune disorder in a subject comprising administering an enhancing agent to the subject.

17. The method of claim 13 or 16, wherein the enhancing agent is a bacterium or is a substance derived from a bacterium.

18. The method of claim 13 or 16, wherein the enhancing agent is administered orally.

19. The method of claim 18, wherein the enhancing agent is a bacterium from the genus Lactobacillus.

20. The method of claim 13 or 16, wherein the enhancing agent is derived from a bacterium belonging to a genus selected from the group consisting of: Mycobacteria, Bordatella, Corynebacterium, Streptococcus, or Hemophilus.

21. The method of claim 20, wherein the enhancing agent is administered orally.

22. The method of claim 20, wherein the enhancing agent is lipopolysaccharide.

23. The method of claim 20, wherein the enhancing agent is in the form of a bacterial cell lysate.

24. The method of claim 20, wherein the enhancing agent is a purified or recombinant bacterial antigen.

25. The method of claim 20, wherein the enhancing agent is lipo-arabinomannan (LAM).

26. The method of claim 20, wherein the enhancing agent is an α- galactosyl-ceramide.

27. The method of claim 13 or 16, wherein the autoimmune disorder is selected from the group consisting of: hay fever, allergic rhinitis, and asthma.

28. A kit for predicting the propensity of a subject to develop an autoimmune disorder or the effectiveness of a treatment for an autoirnmune disorder comprising a detection reagent selected from the group consisting of: at least one antibody which recognizes a cell surface marker on an indicator cell and a probe that recognizes a nucleic acid molecule present in an indicator cell.

29. The kit of claim 28, further comprising at least one detection reagent that recognizes a cytokine.

30. The kit of claim 28, further comprising a means for isolating peripheral blood mononuclear cells.
